(19) United States
(12) Patent Application Publication
(10) Pub. No.: US 2010/0004742 A1
(43) Pub. Date: Jan. 7, 2010

(54) Multiocular Intraocular Lens System

(75) Inventor: J. Stuart Cumming, Laguna Beach, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(73) Assignee: C7C VISION INTERNATIONAL LIMITED

(21) Appl. No.: 12/486,588

(22) Filed: Jun. 17, 2009

Related U.S. Application Data
(63) Continuation-in-part of application No. 11/623,655, filed on Jan. 16, 2007, now abandoned.
(60) Provisional application No. 60/822,475, filed on Aug. 15, 2006.

(51) Int. Cl. A61F 2/16

(52) U.S. Cl. 623/6.27; 623/6.11

(57) ABSTRACT

An accommodating intraocular lens having anteriorly and posteriorly movable extended portions, such as T-shaped haptics, extending from a central optic to be implanted within a human eye, and a second optic spaced from the posterior optic. The first optic is intended to be implanted in the capsular bag, and the second optic may be located in the capsular bag, in the sulcus, or in the anterior chamber. The second optic can be spaced from and fixed to the first optic and this lens assembly implanted in the capsular bag.

### BACKGROUND OF THE INVENTION

This application claims the benefit of U.S. Provisional Application No. 60/822,475 filed Aug. 15, 2006, which applications are incorporated herein by reference, and is a continuation-in-part of application Ser. No. 11/623,655.

This invention relates generally to intraocular lenses to be implanted within the human eye formed by evacuation of the crystalline matrix from the natural lens of the eye through an anterior capsulotomy in the lens. The invention relates more particularly to novel accommodating intraocular lenses of this kind having a number of improved features including, most importantly, increased depth of focus.

The human eye has an anterior chamber between the cornea and iris, a posterior chamber behind the iris containing a crystalline lens, a vitreous chamber behind the lens containing vitreous humor, and a retina at the rear of the vitreous chamber. The crystalline lens of a normal human eye has a lens capsule attached about its periphery to the ciliary muscle of the eye by zonules and containing a crystalline lens matrix. This lens capsule has elastic optically clear anterior and posterior membrane-like walls commonly referred to by ophthalmologists as anterior and posterior capsules, respectively. Between the iris and the ciliary muscle is an annular crevice-like space called the ciliary sulcus.

The young human eye possesses natural accommodation capability. Natural accommodation capability involves relaxation and contraction of the ciliary muscle of the eye by the brain to provide the eye with near and distant vision. This ciliary muscle action is automatic and shapes the natural crystalline lens to the appropriate optical configuration for focusing on the retina the light rays entering the eye from the scene being viewed.

The human eye is subject to a variety of disorders which degrade or totally destroy the ability of the eye to function properly. One of the more common of these disorders involves progressive clouding of the natural crystalline lens matrix resulting in the formation of what is referred to as a cataract. It is now common practice to cure a cataract by surgically removing the cataractous human crystalline lens and implanting an artificial intraocular lens in the eye to replace the natural lens. The prior art is replete with a vast assortment of intraocular lenses for this purpose.

Intraocular lenses differ widely in their physical appearance and arrangement. This invention is concerned with intraocular lenses of the kind having a central optical region or optics and haptics which extend outward from the optics and engage the interior of the eye in such a way as to support the optic on the axis of the eye.

Intraocular lenses differ with respect to their accommodation capability, and their placement in the eye. Accommodation is the ability of an intraocular lens to accommodate, that is, to focus the eye for near and distant vision. Certain patents describe alleged accommodating intraocular lenses. Other patents describe non-accommodating intraocular lenses. Most non-accommodating lenses have single focus optics which focus the eye at a certain fixed distance only and require the wearing of eye glasses to change the focus. Other non-accommodating lenses have multifocal optics which image both near and distant objects on the retina of the eye. The brain selects the appropriate image and suppresses the other image so that a multifocal intraocular lens provides both near vision and distant vision sight without eyeglasses. Bifocal intraocular lenses, however, suffer from the disadvantage that each bifocal image represents only about 40% of the available light, and a remaining 20% of the light is lost in scatter.

There are four possible placements of an intraocular lens within the eye. These are (a) in the anterior chamber, (b) in the posterior chamber, (c) in the capsular bag, and (d) in the vitreous chamber. The intraocular lenses disclosed herein are mainly for placement in the capsular bag but some are placed in the sulcus and/or the anterior chamber.

### SUMMARY OF THE INVENTION

This invention provides an improved accommodating intraocular lens to be implanted within a human eye which remains intact within the eye after removal of the crystalline lens matrix from the natural capsule of the lens of the eye through an anterior capsule opening in the natural lens. This anterior opening is created by performing an anterior capsulotomy, preferably an anterior capsulorhexis, on the natural lens and is circumferentially surrounded by an anterior capsular rim which is the remnant of the anterior capsule of the natural lens. An improved accommodating intraocular lens according to the invention includes one or more central optics having normally anterior and posterior sides and extended portions spaced circumferentially about and extending generally radially out from the edge of the optic. These extended portions have inner ends joined to the optic and opposite outer ends movable anteriorly and posteriorly relative to the optic. To this end, the extended portions are either pivotally or flexibly hinged at their inner ends to the optic or are resiliently bendable throughout their length. In this disclosure, the terms “flex”, “flexing”, “flexible”, and the like are used in a broad sense to cover both flexibly hinged and resiliently bendable extended portions. The terms “hinge”, “hinged”, “hinging”, and the like are used in a broad sense to cover both pivotally and flexibly hinged extended portions.

The lens is surgically implanted within a patient's eye through the anterior capsule opening in the bag and in a position wherein the lens optic is aligned with the opening, and the outer ends of the lens extended portions are situated within the outer perimeter or cul-de-sac of the bag, or in the sulcus or anterior chamber. The lens has a radial dimension from the outer end of each extended portion to the axis of the lens optic such that when the lens is implanted within the eye, the outer ends of the extended portions engage an inner perimetrical wall.

After surgical implantation of the accommodating intraocular lens in the capsular bag of the eye, active ectodermal cells on the posterior side of the anterior capsule rim of the bag cause fusion of the rim to the elastic posterior capsule of the bag by fibrosis. This fibrosis occurs about the lens extended portions in such a way that these extended portions are effectively “shrink-wrapped” by the fibrous tissue in such a way as to form radial pockets in the fibrous tissue which contain the extended portions with their outer ends positioned within the outer cul-de-sac of the capsular bag. In this case, the lens is thereby fixated within the capsular bag with the lens optic aligned with the anterior capsule opening in the bag. The anterior capsule rim shrinks during fibrosis, and this shrinkage combined with shrink-wrapping of the extended portions causes some radial compression of the lens in a manner which tends to move the lens optical system relative to the outer ends of the extended portions posteriorly along the axis of the eye. The fibrosed, leather-like anterior capsule rim prevents anterior movement of the optic and urges the optic rearwardly during fibrosis. Accordingly, fibrosis induced movement of the optic system occurs posteriorly to a distant vision position during the healing process in which either or both the optic and the inner ends of the extended portions press rearwardly against the elastic posterior capsule of the capsular bag and stretch this posterior capsule rearwardly.

Normal brain-induced relaxation and contraction of the ciliary muscle after the completion of fibrosis thus causes anterior and posterior accommodation movement of the lens optical system between near and distant vision positions relative to the retina. During this accommodation movement of the optical system, the lens extended portions undergo endwise movement within their pockets in the capsular bag.

According to another important aspect of this invention, the extended portions of a presently preferred lens embodiment can be generally T-shaped haptics each including a haptic plate and a pair of relatively slender resiliently flexible fixation fingers at the outer end of the haptic plate. In their normal unstressed state, the two fixation fingers at the outer end of each haptic plate extend laterally outward from opposite edges of the respective haptic plate in the plane of the plate and substantially flush with the radially outer end edge of the plate to form the horizontal “crossbar” of the haptic T-shape. The radially outer end edges of the haptic plates are circularly curved about the central axis of the lens optical system to substantially equal radii closely approximating the radius of the interior perimeter of the capsular bag when the ciliary muscle of the eye is relaxed. During implantation of the lens in the bag, the inner perimetrical wall of the bag deflects the haptic fingers generally radially inward from their normal unstressed positions to arcuate bent configurations in which the radially outer edges of the fingers and the curved outer end edges of the respective haptic plates conform approximately to a common circular curvature closely approximating the curvature of the inner perimetrical wall of the bag. The outer T-ends of the haptics then press lightly against the perimetrical bag wall and are fixated within the bag perimeter during fibrosis to accurately center the implanted lens in the bag with the lens optical system aligned with the anterior capsule opening in the bag.

The haptic plates of certain described lens embodiments are narrower in width than the optic diameter. These relatively narrow plates of the haptics flex or pivot relatively easily to aid the accommodating action of the lens and form haptic pockets of maximum length in the fibrosed capsular bag between the haptic fingers and the optic which maximize the accommodation movement of the lens optic. The haptics can slide radially in the capsular bag pockets during contraction of the ciliary muscle to enable forward movement of the optical system for vision accommodation.

In some described lens embodiments of the invention, the lens optical system and extended portions are molded or otherwise fabricated as an integral one piece lens structure in which the inner ends of the extended portions are integrally joined to the optical system, and the extended portions are either resiliently flexible at each point throughout their length or have flexible hinges at their inner ends adjacent the optical system at which the extended portions are hingable anteriorly and posteriorly relative to the optic. In other described lens embodiments, the optics and extended portions are formed separately and have mating hinge portions which interengage to pivotally join an optic and extended portions. In some of these described embodiments, the extended portions are T-shaped haptics formed by molding or otherwise forming the flexible haptic fingers integrally with the haptic plates proper. In other described inventive embodiments, the extended portions are T-shaped haptics having T-shaped reinforcing inserts or inlays which both reinforce the haptic plates and provide the haptics with their T-shapes. Still other described embodiments have reinforcing inserts which reinforce the haptics, provide the haptics with their T-shapes, and/or provide the haptics and optical system with mating pivotal hinge portions for pivotally connecting the haptics to the optical system.

Presently preferred accommodating intraocular lenses of the invention are described. These preferred lenses comprise two optics integrally separated from each other by a fixed space, are generally T-shaped, flexibly hinged haptics and optics whose posterior portions provide most of the optical power of the optics. These optics cooperate with the anteriorly biased configurations of the lenses to increase accommodation amplitude or diopters of accommodation.

### BRIEF DESCRIPTION OF THE DRAWINGS

FIG. 1 diagrammatically illustrates a pair of optics for a multi-ocular system disposed with reference to the cornea and the retina.

FIG. 2 shows an example dual optic lens with haptics extending from one optic.

FIG. 3 is a plan view of the optic of FIG. 2 further illustrating T-shaped haptics.

FIG. 4 is a cross-sectional view showing the optics as well as plural spacers attaching the two optics together.

FIG. 5 is a further view of a posterior lens.

FIG. 6 is a further view of an anterior lens having a larger diameter than the posterior lens.

FIGS. 7a-7b are side and plan views illustrating optics and suitable spacers.

FIGS. 8 through 12 are diagrammatic views illustrating different placements of lenses in the eye with FIG. 8 showing a conventional placement in the capsular bag,

FIG. 9 showing two lenses in the capsular bag, FIG. 10 showing one lens in the capsular bag and one in the sulcus, FIG. 11 showing one lens in the bag and one in the anterior chamber, and FIG. 12 showing two optics integrally linked in the bag.

FIG. 13 shows the lens system in vitro.

FIG. 14 shows the lens system in vitro optic fibrosis.

FIG. 15 illustrates a human eye with a currently available accommodating intraocular lens.

Turning now to these drawings, and first to FIG. 15, there is illustrated a human eye 10 whose natural crystalline lens matrix has been removed from the natural lens capsule of the eye through an anterior opening in the capsule formed by an anterior capsulotomy, in this case a continuous tear circular capsulotomy, or capsulorhexis. As noted earlier, this natural lens matrix, which is normally optically clear, often becomes cloudy and forms a cataract which is cured by removing the matrix and replacing it with an artificial intraocular lens.

Continuous tear circular capsulotomy, or capsulorhexis, involves tearing the anterior capsule along a generally circular tear line in such a way as to form a relatively smooth-edged circular opening in the center of the anterior capsule. The cataract is removed from the natural lens capsule through this opening. After completion of this surgical procedure, the eye includes an optically clear anterior cornea 12, an opaque sclera 14 on the inner side of which is the retina 16 of the eye, an iris 18, a capsular bag 20 behind the iris, and a vitreous cavity 21 behind the capsular bag filled with the gel-like vitreous humor. The capsular bag 20 is the structure of the natural lens of the eye which remains intact within the eye after the continuous tear circular tear capsulorhexis has been performed and the natural lens matrix has been removed from the natural lens.

The capsular bag 20 includes an annular anterior capsular remnant or rim 22 and an elastic posterior capsule 24 which are joined along the perimeter of the bag to form an annular crevice-like cul-de-sac 25 between rim and posterior capsule. The capsular rim 22 is the remnant of the anterior capsule of the natural lens which remains after capsulorhexis has been performed on the natural lens. This rim circumferentially surrounds a central, generally round anterior opening 26 (capsulotomy) in the capsular bag through which the natural lens matrix was previously removed from the natural lens. The capsular hag 20 is secured about its perimeter to the ciliary muscle 28 of the eye by zonules 30.

Natural accommodation in a normal human eye having a normal human crystalline lens involves automatic contraction or constriction and relaxation of the ciliary muscle of the eye by the brain in response to looking at objects at different distances. Ciliary muscle relaxation, which is the normal state of the muscle, shapes the human crystalline lens for distant vision. Ciliary muscle contraction shapes the human crystalline lens for near vision. The brain-induced change from distant vision to near vision is referred to as accommodation.

Implanted within the capsular bag 20 of the eye 10 is an accommodating intraocular lens 32 such as shown in U.S. Pat. No. 7,048,760 which replaces and performs the accommodation function of the removed human crystalline lens. The accommodating intraocular lens may be utilized to replace either a natural lens which is virtually totally defective, such as a cataractous natural lens, or a natural lens that provides satisfactory vision at one distance without the wearing of glasses but provides satisfactory vision at another distance only when glasses are worn. For example, the accommodating intraocular lens of the invention as described below can be utilized to correct refractive errors and restore accommodation for persons in their mid-40s who require reading glasses or bifocals for near vision.

Intraocular lens 32 comprises a unitary body which may be formed of relatively hard material, relatively soft flexible semi-rigid material, or a combination of both hard and soft materials. Examples of relatively hard materials which are suitable for the lens body are methyl methacrylate, polysulfones, and other relatively hard biologically inert optical materials. Examples of suitable relatively soft materials for the lens body are silicone, hydrogels, thermolabile materials, and other flexible semi-rigid biologically inert optical materials.

### DESCRIPTION OF THE PREFERRED EMBODIMENTS

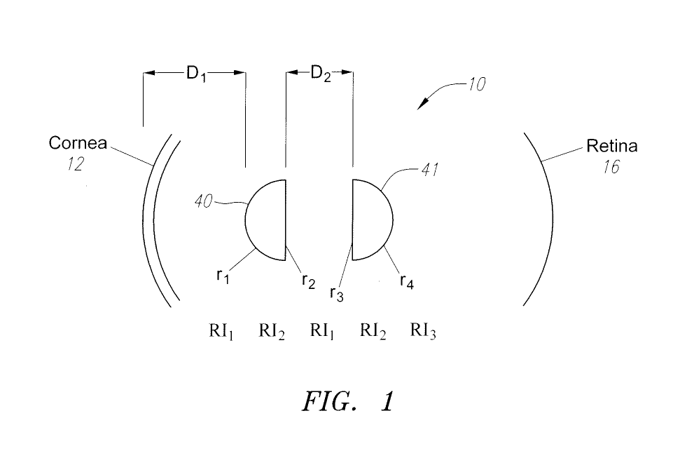

The lens system comprises two optics fused together, one in front of the other, as will be further explained beginning with FIG. 1 below. T-shaped extended portions or plate haptics 36 extend from diametrically opposite edges of the optic. These haptics include haptic members or plates 36 proper having inner ends joined one or other of the optics and opposite outer free ends and lateral fixation fingers at their outer ends. The haptic plates 36 may be longitudinally tapered so as to narrow or widen in width toward their ends or may be wider in their periphery and narrower adjacent to the optic. The optical system 34 is movable anteriorly and posteriorly relative to the haptics 36. The preferred lens embodiment illustrated is constructed of a resilient semi-rigid material and has flexible hinges 38 which join the inner ends of the haptic plates 36 to one of the optics. The haptics are relatively rigid and are flexible about the hinges anteriorly and posteriorly relative to the optic. These hinges are formed by grooves 38 which enter either the anterior or posterior sides and extend across the inner ends of the haptic plates 36. The haptics 36 are flexible about the hinges 38 in the anterior and posterior directions of the optical system. The lens has a relatively flat unstressed configuration, wherein the haptics 36 and their hinges 38 are disposed in a common plane transverse to the optic axis of the optic 34. Deformation of the lens from this normal unstressed configuration by anterior or posterior movement of the haptics about their hinges creates in the hinges elastic strain energy forces which urge the lens to its normal unstressed configuration. The outer end edges of the haptics are preferably circularly curved to equal radii about the optic axis of the optic 34. Anterior movement of the optical system toward the iris also is aided by an increase in vitreous cavity pressure upon constriction of the ciliary muscle. Furthermore this increase in pressure can also deform one or both of the optic further aiding near vision.

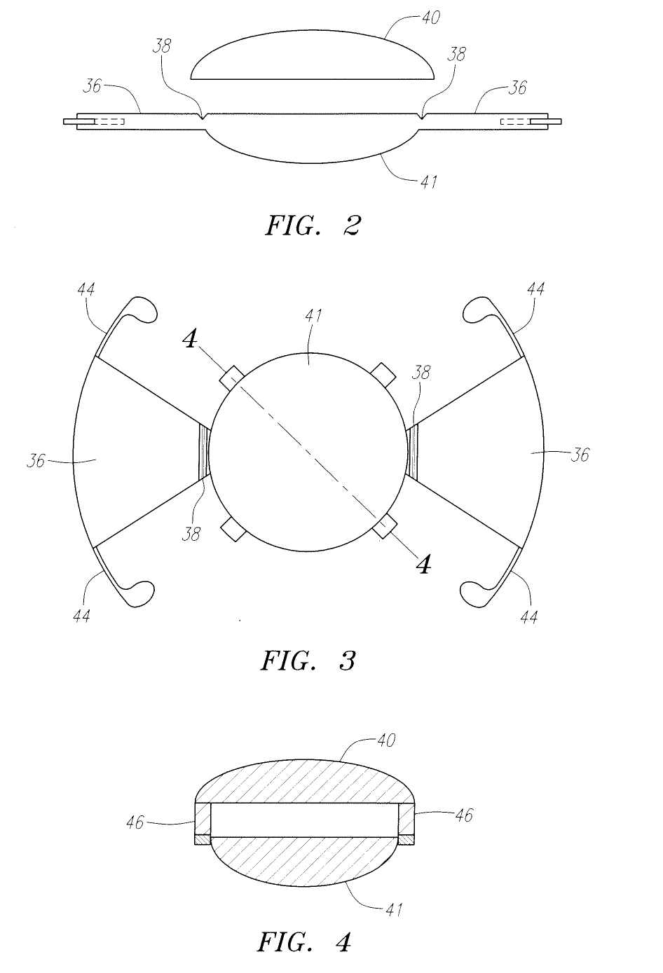

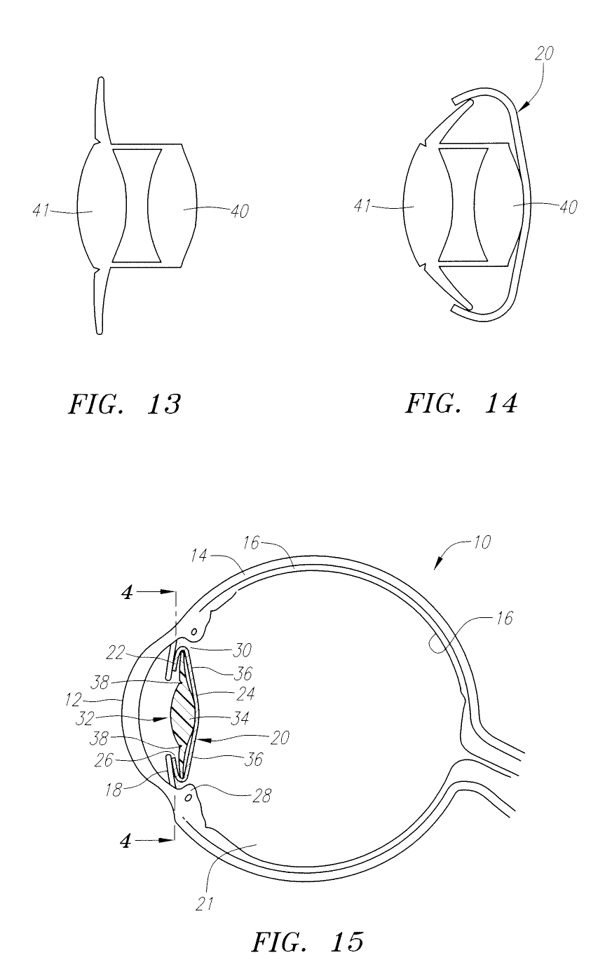

Turning now to FIG. 1, the same diagrammatically illustrates the human eye 10, the cornea 12, the retina 16, and further including an anterior optic 40 and posterior optic 41. Although not shown in FIG. 1, normally the posterior optic 41 includes haptics 36 such as seen in FIGS. 2 and 3 (and FIG. 13). D1 represents the distance from the cornea 12 to the first optic 40 and D2 the space between the two optics 40 and 41. D₂ typically ranges from 0 to 3.0 mm. one of the optics can have a torric surface.

The letters “r” represent the four possible radii of the two optics, and they range from 4.9 mm to 6.0 mm. R1, represents the refractive index of the aqueous between the cornea 12 and first optic 40, RI₁ and R12′ represent the refractive indices of respective optics 40 and 41, RI₁′ represents the aqueous between the two optics, and R₃ represents the refractive index of the vitreous between posterior lens 41 and the retina 16. RI₁ is typically 1.336, RI₃ 1.336, and RI₂ 1.427, D₂ is 1.0 to 2.0 mm and typically 1.4 mm. The various radii, refractive indices and distances between the optics can be adjusted to give the greatest depth of focus.

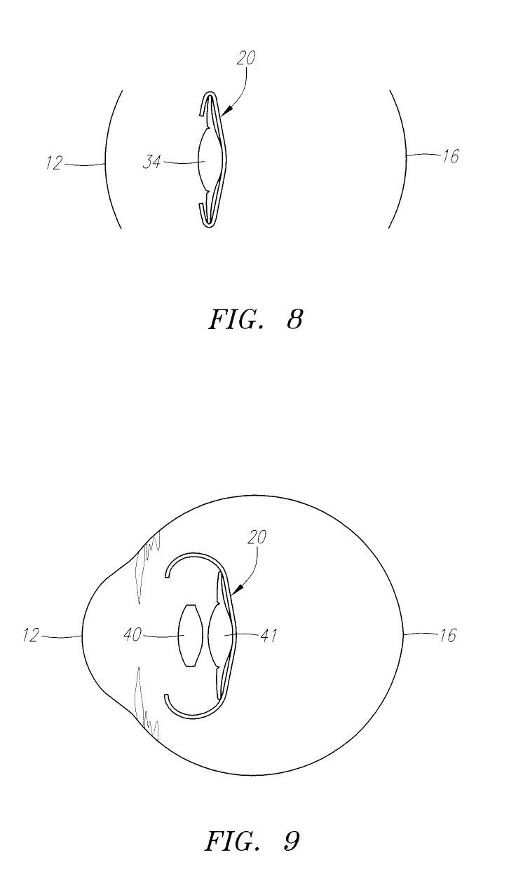

FIG. 2 illustrates the multi-ocular lens system wherein the anterior optic 40 has a larger diameter than the posterior optic 41. The lens has haptics 36 with hinges 38 adjacent the optic 41. FIG. 3 is a plan view of the posterior optic 41 illustrating T-shaped haptics 36, hinges 38 adjacent the optic, and fixation fingers 44. FIG. 4 illustrates the manner in which the two optics 40 and 41 are spaced and can be sealed with posts 46, preferably with liquid silicone and heat. The design is such that the anterior optic 40 can attach to the posterior lens 41. As can be seen from FIGS. 9-11, the anterior optic 40 can have haptics and fixation fingers like lens 41.

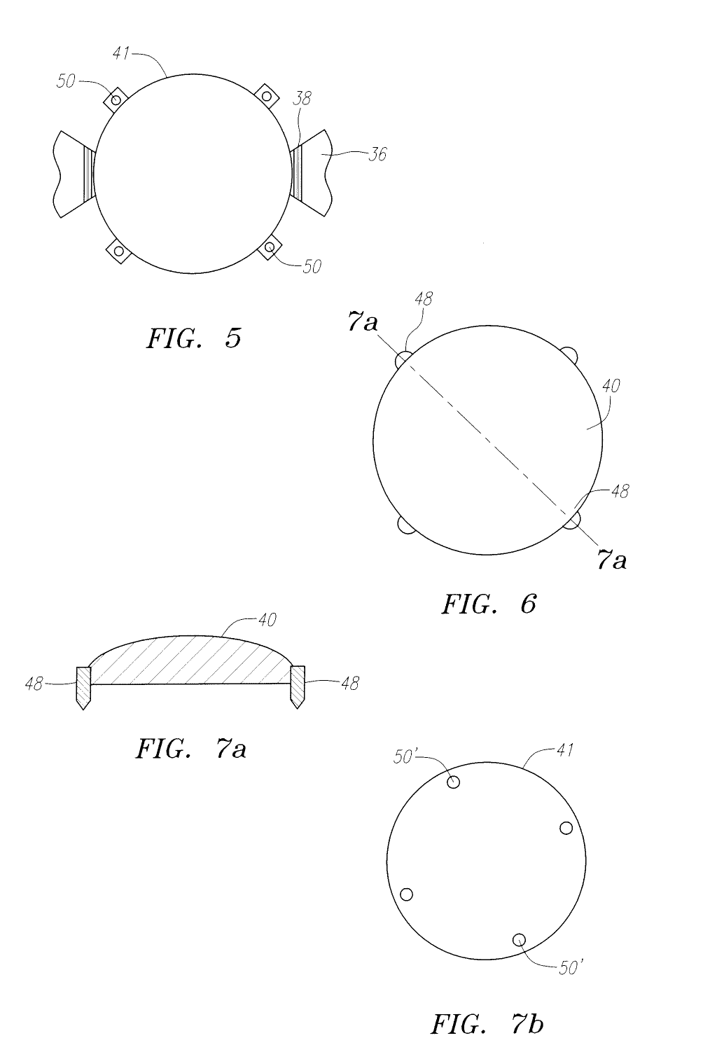

FIGS. 5 through 7b illustrate the posterior lens 41, anterior optic 40, and stakes 48, via which the anterior optic can be connected with suitable holes 50 or 50′ as seen in FIGS. 5 and 7b. The two optics 40 and 41 can be attached before implantation or after implantation. The anterior optic 40 can be detachable so that it can be changed after implantation to provide a power change or a torricity charge.

The lens 41 can have an optic diameter of 4.0-6.5 mm, length from haptic 36 end to end of 10.0-12.5 mm, loop 44 tip to loop tip 10.5-13.0 mm, hinge 38 width 1.0-5.0 mm and depth at base of 0.05-1.0 mm. Typical materials are silicone, acrylic or any suitable optical material, and polymide or other logs material such as PMAA.

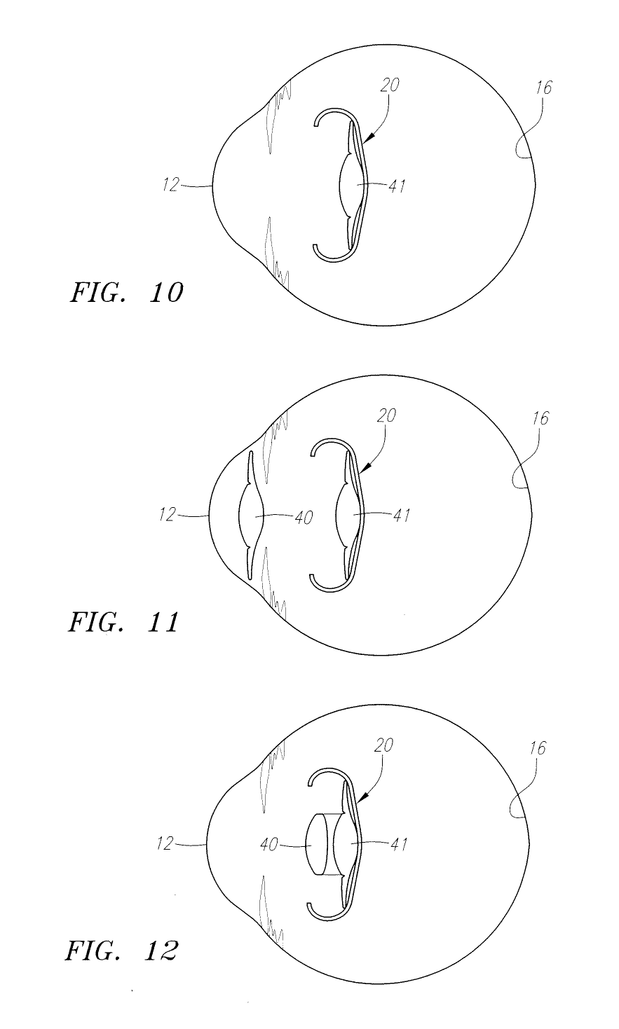

Turning now to FIGS. 8 through 12, FIG. 8 is a schematic representation similar to FIG. 13 showing an optic 34 of a standard intraocular lens in the capsular bag 20. FIG. 9 diagrammatically illustrates both lenses 40 and 41 with haptics disposed in the capsular bag. FIG. 10 diagrammatically illustrates optic 41 in the capsular bag 20 and the anterior optic 40 in the sulcus.

FIG. 11 diagrammatically illustrates two individual lenses 41 in the capsular bag 20, and the lens 40 in the anterior chamber. FIG. 12 illustrates the lens system 40 and 41 integrally linked and disposed in the capsular bag. In each case, the posterior optic can be standard accommodating intraocular lens.

Either lens 40 or 41 can be a stabilized accommodating intraocular lens according to patent application Ser. No. 11/461,290 filed Jul. 31, 2006, Attorney Docket No. 13533.4069.

FIG. 13 shows the lens system in vitro. The lens system may be designed such that the haptics are attached to the anterior optic resulting in an anterior vault when the lens system is focused for distance as in FIG. 14 or to the posterior optic resulting in a posterior vault when the lens system is in the distance position. FIG. 14 shows the lens in vitro after fibrosis

While an embodiment of the present invention has been shown and described, various modifications may be made without departing from the scope of the present invention, and all such modifications and equivalents are intended to be covered.

1. An accommodating intraocular lens comprising a first flexible optic having anterior and posterior sides; and haptics comprising at least two portions extending from the optic, the portions having inner ends adjacent the optic and outer ends distal to the optic, and the portions having fixation members at the outer ends of the portions, and
a second optic spaced at a fixed distance from and attached to the first optic and integrally part of the optical system the two optics being movable anteriorly and posteriorly together relative to the outer ends of the haptics.

2. The intraocular lens of claim 1, wherein the portions are haptic plates.

3. The intraocular lens of claim 1, wherein each portion comprises a plate haptic with at least one finger at the distal end of the portion.

4. The intraocular lens of claim 1, wherein the portions are resiliently bendable throughout a portion of their length.

5. The intraocular lens of claim 1, wherein the first optic, portions, and fixation members are integrally formed.

6. The intraocular lens of claim 1, wherein the second optic portion, and fixation members are integrally formed.

7. The intraocular lens of claim 1, further comprising a hinge between the inner ends of the portions and the optic to which the haptic is attached.

8. The intraocular lens of claim 1, wherein the portions include a thinned area thereby forming a hinge.

9. The intraocular lens of claim 8, wherein the flexible hinges are formed by a groove.

10. The intraocular lens of claim 1, wherein said portions and fixation members comprise T-shaped haptics.

11. The intraocular lens of claim 1 wherein the first optic is a posterior optic and the second optic is an anterior optic.

12. The intraocular lens of claim 1 wherein the first optic is a anterior optic and the second optic is an posterior optic.

13. The intraocular lens of claim 10 wherein the first and second optics are to be implanted in the capsular bag.

14. The intraocular lens of claim 10 wherein the first optic is to be implanted in the capsular bag and the second optic lies within the sulcus.

15. The intraocular lens of claim 10 wherein the first optic is to be implanted in the capsular bag and the second optic is to be implanted in the anterior chamber.

16. An accommodating intraocular lens comprising a first flexible optic having anterior and posterior sides, and haptics comprising at least two portions extending from the optic, the portions having inner ends adjacent the optic and outer ends distal to the optic, and the portions having fixation members at the outer ends of the portions, and
a second optic spaced at a fixed distance from and attached to the first optic, the two optics being movable together anteriorly and posteriorly relative to the outer ends of the haptics.

17. The intraocular lens of claim 15 wherein the lens is for implantation in the capsular bag.
